(19) United States
(12) Patent Application Publication
(10) Pub. No.: US 2021/0244263 A1
(43) Pub. Date: Aug. 12, 2021

(54) COIL SHEATH AND MEDICAL DEVICE

(71) Applicants: nano grains Co., Ltd., Suwa-shi (JP); Mikuro Spring Co., Ltd., Suwa City (JP)

(72) Inventors: Keita Suzuki, Chino-shi (JP); Makoto Natori, Suwa City (JP); Yusuke Sato, Suwa City (JP)

(73) Assignees: nano grains Co., Ltd., Suwa-shi (JP); Mikuro Spring Co., Ltd., Suwa City (JP)

(21) Appl. No.: 17/172,891

(22) Filed: Feb. 10, 2021

(30) Foreign Application Priority Data
Feb. 12, 2020 (JP) 2020-021587

(51) Int. Cl. A61B 1/00; A61B 1/005

(52) U.S. Cl. CPC A61B 1/00087; A61B 1/0051

(57) ABSTRACT

According to one embodiment, a coil sheath is provided which is formed by spirally winding a wire and having a plurality of unit loops arranged side by side in a direction of an axis, wherein each unit loop includes a plurality of non-circular loops each having a long axis portion and a short axis portion, a dimension of the long axis portion is longer than a short axis portion of the other loops, and, in front view seen in an axial direction, each unit loop includes a central space including the axis and a plurality of peripheral spaces located around the central space.

### CROSS-REFERENCE TO RELATED APPLICATIONS

This application claims priority to and the benefit of Japanese Patent Application No. 2020-021587, filed on Feb. 12, 2020, the entire contents of which are incorporated herein by reference in their entirety.

### FIELD

Embodiments described herein relate generally to a coil sheath and a medical device provided with the coil sheath.

### BACKGROUND

In endoscopes and treatment tools for endoscopes, a coil sheath is widely used as a member constituting a long portion (insertion portion) to be inserted into the body. The coil sheath is formed by spirally winding a wire made of metal or resin. (See, for example, Japanese Unexamined Patent Application, First Publication No. 2000-229084.)

In endoscopes and treatment tools for endoscopes, there is a need to enable the insertion portion to be bent in the body. As a configuration that makes this possible, a configuration is known in which a plurality of cylindrical members such as curved pieces and knots are arranged and an angle wire is passed through a minute hole formed in the cylindrical member.

However, since this configuration is complicated, it is difficult to apply it to an endoscopic treatment tool having an outer diameter of about several millimeters.

### BRIEF SUMMARY

An embodiment of the present invention provides a coil sheath capable of easily realizing a configuration in which a bending operation can be performed.

A first aspect of the present invention is a coil sheath formed by spirally winding a wire and having a plurality of unit loops arranged side by side in a direction of an axis.

In this coil sheath, each unit loop includes a plurality of non-circular loops each having a long axis portion and a short axis portion. A dimension of the long axis portion is longer than a short axis portion of the other loops, and in front view seen in an axial direction, each unit loop includes a central space including the axis and a plurality of peripheral spaces located around the central space.

A second aspect of the present invention is a medical device including an elongated insertion portion.

In this medical device, at least a part of the insertion portion is composed of the coil sheath according to the present invention, and includes an operation wire that passes through the peripheral space.

According to the coil sheath of an embodiment of the present invention, a configuration capable of bending can be easily realized.

### BRIEF DESCRIPTION OF DRAWINGS

FIG. 1 is a diagram showing a coil sheath according to the first embodiment of the present invention.

FIG. 2 is a front view showing a unit loop of the coil sheath.

FIG. 3 is an enlarged view showing a distal end portion of an endoscopic treatment tool to which the coil sheath is applied.

FIG. 4 is a cross-sectional view taken along the line I-I of FIG. 3.

FIG. 5 is a front view showing a unit loop of a coil sheath according to a second embodiment of the present invention.

FIG. 6 is a front view showing a unit loop of a coil sheath according to a modified example of the present invention.

FIG. 7 is a front view showing one of the loops constituting the unit loop.

FIG. 8 is an example of a side view of a modified coil sheath.

FIG. 9 is another example of a side view of a modified coil sheath.

### DETAILED DESCRIPTION

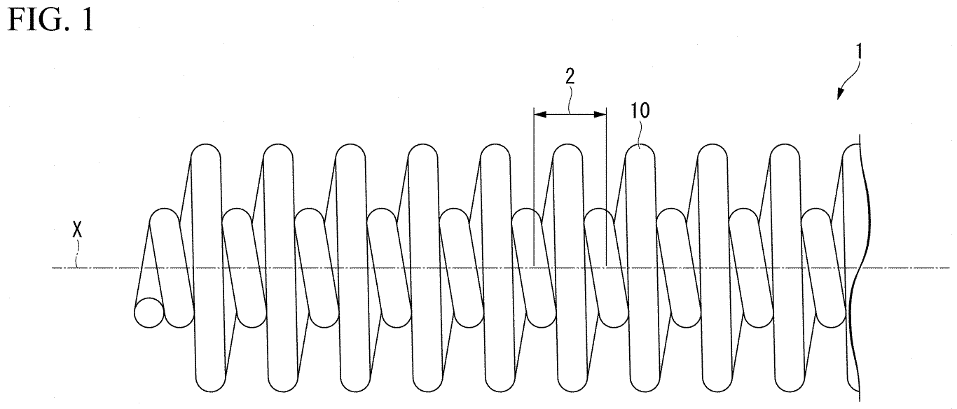

A first embodiment of the present invention will be described with reference to FIGS. 1 to 4. FIG. 1 is a schematic view showing a coil sheath 1 according to the present embodiment. The coil sheath 1 is formed by spirally winding strands 10 while forming a unit loop 2. The coil sheath 1 has a configuration in which a plurality of unit loops 2 are repeatedly arranged in the X direction of the axis.

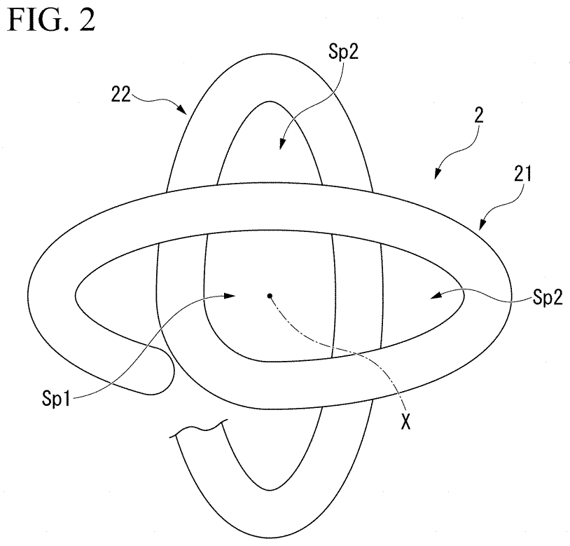

FIG. 2 shows the front view (state of the unit loop 2 in the extending direction of the axis X). The unit loop 2 has a non-circular first loop 21 and a second loop 22. In the present embodiment, the first loop 21 and the second loop 22 have the same shape and are elliptical having a long axis and a short axis. However, the non-circular shape may have a long portion (long axis portion) and a short portion (short axis portion) in the direction orthogonal to the extending direction of the axis X, and is not limited to the elliptical shape. Therefore, it does not have to be a mathematical ellipse, and may be an oval or the like in which a part of the loop is linear.

The first loop 21 and the second loop 22 are arranged in a coaxial shape (including substantially coaxial; the same applies hereinafter) in which the centers are aligned in a front view, and are in a positional relationship in which their long axes are approximately 90 degrees. Thereby, the central portions of the first loop 21 and the second loop 22 overlap each other in the front view, but both ends in the long axis direction do not overlap, and the first loop 21 and the second loop 22 are positioned around the central portion at approximately equal intervals of 90 degrees of rotation angles.

The coil sheath 1 includes a central space Sp1 located around the central space Sp1 including the axis X by having a large number of unit loops 2 continuous in the axis X direction, and four peripheral spaces Sp2 arranged at equal intervals around the central space Sp1.

When manufacturing the coil sheath 1, the wire 10 is wound so as to alternately form the first loop 21 and the second loop 22. For example, the first loop 21 starts to be wound from the vicinity of the point where the first loop 21 and the second loop 22 overlap in the front view, and when the first loop 21 is formed by winding one round, the direction is changed and the second loop 22 is wound. As a result, one unit loop 2 is formed, and by repeating this a desired number of times, a coil sheath 1 having a desired length can be manufactured. At the end of the coil sheath, only a part of the loops (first loop 21 or second loop 22) constituting the unit loop may be present as a remainder.

Examples of the material of the wire 10 include metal and resin, and among them, various metals are preferable.

Although the coil sheath 1 of the present embodiment can be formed only by winding the wire 10, it has a plurality of peripheral spaces Sp2 isolated from the central space Sp1 around the central space Sp1. Therefore, by passing an angle wire (operation wire) through the peripheral space, a small diameter structure that can be bent can be manufactured. The production of a curved structure using the coil sheath 1 is remarkably simpler than the conventional method of forming and connecting minute holes in a plurality of knot rings or the like, and the diameter can be easily reduced.

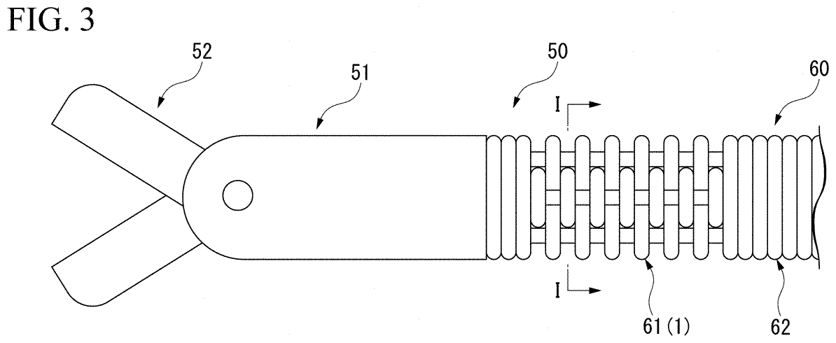

FIG. 3 shows an example of an endoscopic treatment tool to which the coil sheath 1 is applied.

FIG. 3 is an enlarged view showing the distal end of the forceps 50 used through the treatment tool channel of the endoscope. The forceps 50 includes a treatment portion 51 having a pair of forceps pieces 52, an elongated insertion portion 60 connected to the treatment portion, and an operation portion (not shown) connected to the insertion portion 60. In the insertion portion 60, the portion closer to the treatment portion 51 is a curved portion 61 formed by the coil sheath 1. The insertion portion between the coil sheath 1 and the operation portion is composed of a general coil sheath 62 having a continuous circular loop.

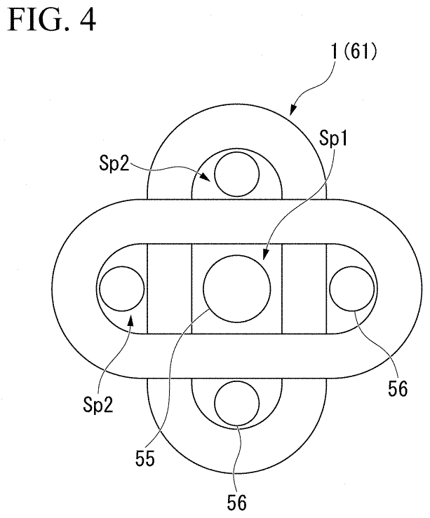

FIG. 4 is a cross-sectional view taken along the line I-I of FIG. 3. An opening/closing wire 55 passes through the central space Sp1 of the coil sheath 1. The front end of the opening/closing wire 55 is connected to a pair of forceps pieces 52, and the rear end is connected to a slider or the like provided in the operation portion. By operating the slider or the like, the opening/closing wire can be moved back and forth in the insertion portion 60 to open/close the pair of forceps pieces 52.

An angle wire (operation wire) 56 is passed through each of the four peripheral spaces Sp2. The front end of each angle wire 56 is fixed to the distal end portion or the treatment portion 51 of the insertion portion 60, and the rear end extends to the operation portion and is connected to an operation mechanism (not shown) such as a dial. When one of the angle wires 56 is pulled, the curved portion 61 is bent toward the peripheral space Sp2 through which the pulled angle wire is passed.

Although FIG. 3 shows an example of a treatment tool for an endoscope, the coil sheath according to the present invention can also be applied to an endoscope. In particular, it is suitable for forming a curved portion of an endoscope having a small diameter (for example, a diameter of 3 mm or less).

A second embodiment of the present invention will be described. In the following description, the same reference numerals will be given to the configurations common to those already described, and duplicate description will be omitted.

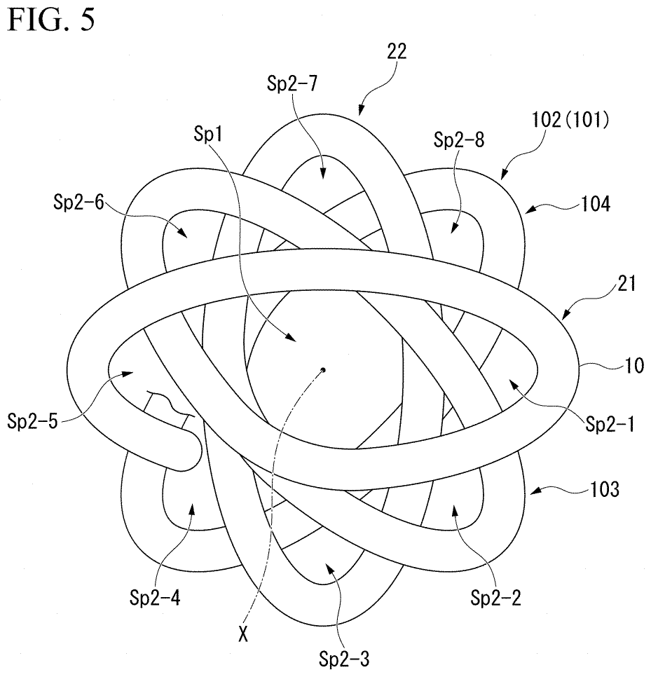

FIG. 5 is a front view showing a unit loop 102 in the coil sheath 101 of the present embodiment. The unit loop 102 has a third loop 103 and a fourth loop 104 in addition to the first loop 21 and the second loop 22, and is composed of four loops. The third loop 103 and the fourth loop 104 have the same front view shape as the first loop 21 and the second loop 22, and are in a positional relationship in which the long axis directions thereof are approximately 90 degrees. The first loop 21 and the third loop 103 are in a positional relationship in which the long axis directions of the first loop 21 and the third loop 103 are approximately 90 degrees.

In the unit loop 102, the strand 10 is wound in the order of the first loop 21, the third loop 103, the second loop 22, and the fourth loop 104. As a result, the coil sheath 101 has a configuration in which eight peripheral spaces Sp2 are arranged at substantially equal intervals around the central space Sp1.

Similarly to the first embodiment, the coil sheath 101 of the present embodiment can easily form a curved portion by passing an angle wire through the peripheral space Sp2. When the angle wires are passed through all the peripheral spaces, the coil sheath 101 can be bent in 8 directions by pulling one of the angle wires. When the angle wires are arranged in every other 4 peripheral spaces, the coil sheath 101 can also be bent in the same manner as the first embodiment.

The coil sheath 101 can exert a function other than bending. When forming a curved portion, the angle wire is passed through the peripheral space of the same loop in the unit loop, but in this case, the peripheral space through which the wire passes is sequentially changed to another adjacent peripheral space.

For example, in FIG. 5, after the wire is passed through the peripheral space Sp2-1 formed by the first loop 21, the wire is then passed through the peripheral space Sp2-2 formed by the third loop 103 adjacent to the peripheral space Sp2-1. After that, by the same procedure, the wire is sequentially passed through the adjacent peripheral spaces Sp2-3, Sp2-4 and Sp2-8, and while repeating this, the wire is extended to the operation portion.

The wires arranged in this way are rotating wires (operation wires) spirally arranged outside the central space Sp1 of the coil sheath 101. In the same manner as this rotating wire, if another rotating wire is arranged in a positional relationship symmetrical with respect to the axis X (in this case, the wires are sequentially passed from Sp2-5), a rotating portion that can be rotated can be configured by using the coil sheath 101. In the rotating portion, the front end of each rotating wire is fixed to the distal end portion of the insertion portion 60 or the treatment portion 51 as in the first embodiment.

When the two rotating wires are towed at the same time in the operation portion, each rotating wire tries to become a straight line parallel to the axis X from the spirally arranged state. As a result, the peripheral space through which the rotating wire is passed moves from a state in which the peripheral space is sequentially displaced around the axis X to a substantially same position around the axis X. Along with this, each loop of the unit loop 102 rotates, and the coil sheath 101 rotates by the total amount of rotation of each loop. As a result, the treatment portion 51 and the like connected to the coil sheath 101 can be rotated.

Conventionally, it has been very difficult to rotate the treatment portion provided at the distal end of an endoscopic treatment tool. The main reason for this is that the torque applied on the hand side is not sufficiently transmitted to the distal end due to the long insertion portion.

In the rotating portion formed by using the coil sheath 101 of the present embodiment, the amount of rotation of each loop in the unit loop 102 is very small, so that the rotation is less likely to be attenuated or lost in each loop. Therefore, stable rotation operation is exhibited even by the torque applied by the operation portion. Further, by increasing the length of the coil sheath 101, the total amount of rotation (integrated amount) of each loop can be easily increased, so that a relatively large amount of rotation such as 180 degrees around the axis X can be easily realized. In a case where it is desired to increase the integrated amount in relation to the rotation amount, the entire insertion portion of the medical device may be the coil sheath of the present invention.

In the above description, a pair of rotating wires are arranged, but when only one rotating wire is arranged and the rotating wire is towed, the coil sheath is bent while rotating. Even when one of the pair of rotating wires is free and only the other is towed, the same behavior is exhibited. In this way, it is also possible to use the coil sheath 101 to form a portion that performs both rotating and bending operations.

Although each embodiment of the present invention has been described above, the technical scope of the present invention is not limited to each of the above embodiments, and various modifications can be made without departing from the spirit of the present invention. Some changes are illustrated below, but these are not all and other changes are possible. These changes may be combined as appropriate.

The number and arrangement of unit loops are not limited to those described in the above embodiment, and can be appropriately set.

The shape of the plurality of loops constituting the unit loop may have a long axis portion and a short axis portion, and a peripheral space may be formed, and the plurality of loops may not be completely the same. In order for the peripheral space to be formed, the dimension of the long axis portion of the loop may be longer than the short axis portion of other adjacent loops.

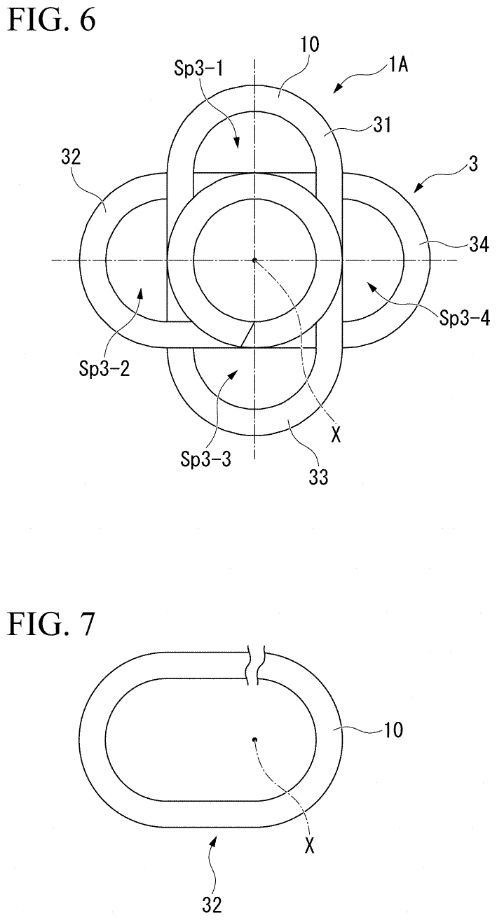

The unit loop 3 of the coil sheath 1A of the modified example shown in FIG. 6 is composed of four loops of a first loop 31, a second loop 32, a third loop 33, and a fourth loop 34. FIG. 7 shows a front view of the second loop 32. The front view shape of the second loop 32 is oval, but by being arranged asymmetrically with respect to the axis X, the peripheral space Sp3-2 is generated in only one direction around the axis X. Each of the other loops 31, 33, and 34 has a frontal view shape of the same shape and size as the second loop 32, and the peripheral spaces Sp3-1, Sp3-3, and Sp3-4 are generated in different directions around the axis, respectively.

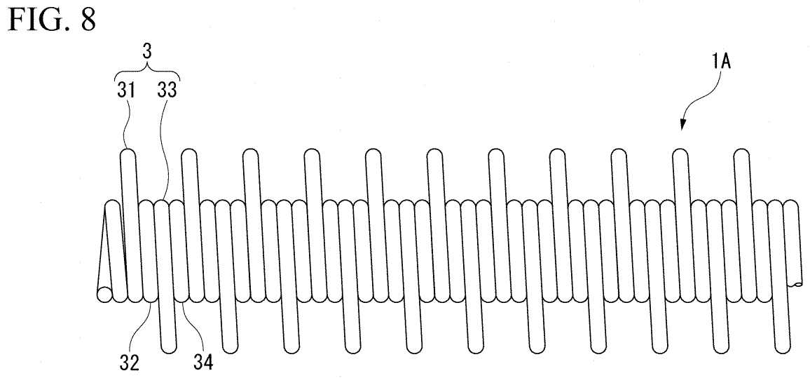

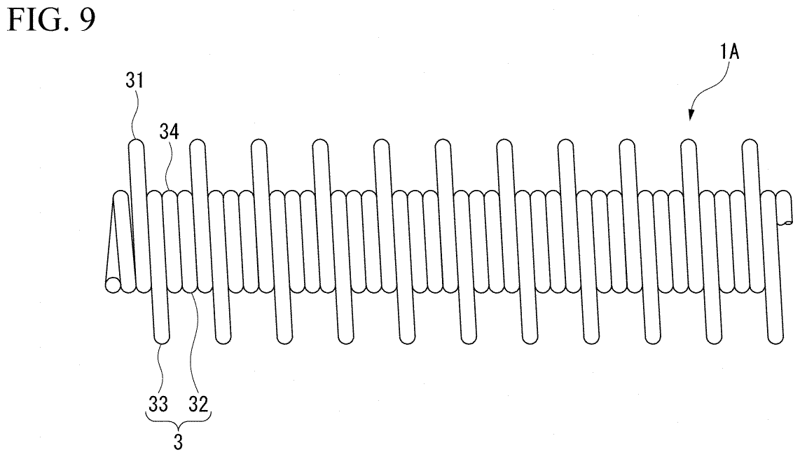

The order of formation of the four loops in the unit loop 3 is not particularly limited and can be appropriately determined. FIG. 8 shows a side view of the coil sheath 1A having the unit loop 3 formed in the order of the first loop 31, the second loop 32, the third loop 33, and the fourth loop 34. FIG. 9 shows a side view of the coil sheath 1A having the unit loop 3 formed in the order of the first loop 31, the third loop 33, the fourth loop 34, and the second loop 32. Although the loop formation order is different between the two, when operated by passing an angle wire or a rotating wire through the peripheral space, they behave substantially in the same manner and have the same effect.

The endoscopic treatment tool to which the coil sheath of the present invention is applied is not limited to the forceps described above. It can be applied to any medical device that has the advantage of bending the distal end and rotating the treatment portion.

In a coil sheath formed by spirally winding a wire and having a plurality of unit loops 2 arranged side by side in the X direction of the axis, the unit loop 2 has a non-circular first loop 21 and a second loop 22 having a long axis portion and a short axis portion. The dimensions of the long axis portion are longer than the short axis portion of the other loops. When viewed from the front in the direction of the axis X, it has a central space Sp1 including the axis X and a plurality of peripheral spaces Sp2 located around the central space Sp1.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A coil sheath formed by spirally winding a wire and having a plurality of unit loops arranged side by side in a direction of an axis,
wherein each unit loop includes a plurality of non-circular loops each having a long axis portion and a short axis portion,
a dimension of the long axis portion is longer than a short axis portion of the other loops, and
in front view seen in an axial direction, each unit loop includes a central space including the axis and a plurality of peripheral spaces located around the central space.

2. The coil sheath according to claim 1, wherein the peripheral spaces are arranged around the axis at equal intervals.

3. A medical device including an elongated insertion portion, wherein at least a part of the insertion portion is composed of the coil sheath according to claim 1, and includes an operation wire that passes through the peripheral space.
